(19) United States
(12) Patent Application Publication
(10) Pub. No.: US 2017/0217952 A1
(43) Pub. Date: Aug. 3, 2017

(54) CRYSTALLINE FORM

(71) Applicant: Gilead Sciences, Inc., Foster City, CA (US)

(72) Inventors: Chiajen Lai, Livermore, CA (US); Lok Him Lawrence Yu, Foster City, CA (US); Richard Hung Chiu Yu, San Francisco, CA (US)

(21) Appl. No.: 15/414,438

(22) Filed: Jan. 24, 2017

Related U.S. Application Data
(60) Provisional application No. 62/288,029, filed on Jan. 28, 2016.

(51) Int. Cl. C07D 417/14

(52) U.S. Cl. CPC C07D 417/14; C07B 2200/13

(57) ABSTRACT

The invention provides a crystalline form of the compound of formula (Ia), methods for making the crystalline form of the compound of formula (Ia), and therapeutic methods for the use of the crystalline form of the compound of formula (Ia).

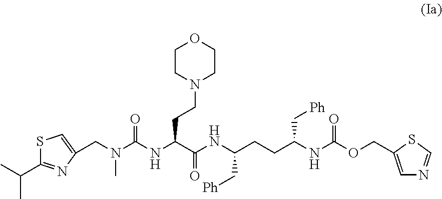 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

### CROSS REFERENCE TO RELATED APPLICATIONS

This non-provisional application claims the benefit of Provisional Application No. 62/288,029, filed Jan. 28, 2016, which is incorporated herein by reference.

### FIELD OF THE INVENTION

The invention provides a crystalline form of the compound of formula (Ia), methods for the preparation of such a form, and therapeutic methods involving the use of such a form.

### BACKGROUND OF THE INVENTION

International patent application PCT/US2007/015604 (published as WO 2008/010921) describes compounds and pharmaceutical compositions which improve the pharmacokinetics of a co-administered drug by inhibiting cytochrome P450 monooxygenase. One such inhibitor is the compound of formula (Ia), the international non-proprietary name for which is cobicistat:

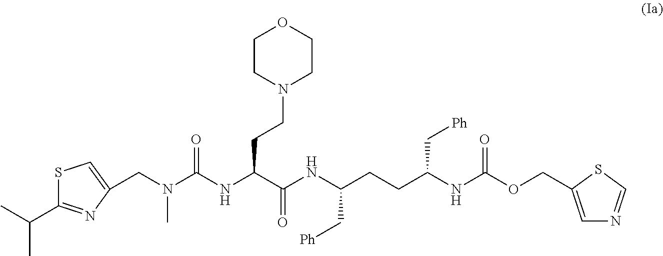 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

In the manufacture of pharmaceutical formulations, it is important that therapeutic agents are in a form which facilitates convenient and economical handling and processing. Accordingly, there is a need for solid forms of therapeutic agents that have beneficial properties, including beneficial physicochemical properties (such as stability, density and hygroscopicity).

### SUMMARY

One embodiment of the invention provides a stable crystalline form of the compound of formula (Ia). In particular, a crystalline form of the compound of formula (Ia), methods for making the crystalline form of the compound of formula (Ia), and therapeutic methods for the use of the crystalline form of the compound of formula (Ia) are provided.

In one embodiment, a crystalline form of the compound of formula (Ia) is provided. In a particular embodiment, the crystalline form is characterised by an X-ray powder diffraction (XRPD) pattern comprising peaks at about (e.g. ±0.5, ±0.3, ±0.2, ±0.1) 17.2 and 19.6 (Cu Kα radiation, expressed in degrees 2θ). In a further embodiment, the crystalline form is characterised by an X-ray powder diffraction (XRPD) pattern comprising at least two, three or four peaks at about (e.g. ±0.5, ±0.3, ±0.2, ±0.1) 13.5, 17.2, 19.6 and 20.8 (Cu Kα radiation, expressed in degrees 2θ). In a further embodiment, the crystalline form is characterised by an X-ray powder diffraction (XRPD) pattern comprising at least two, three, four, five, six or seven peaks at about (e.g. ±0.5, ±0.3, ±0.2, ±0.1) 7.0, 13.5, 14.0, 17.2, 19.6, 20.2, 20.8 and 21.0 (Cu Kα radiation, expressed in degrees 2θ).

In further embodiments, the crystalline form is characterised by an XRPD pattern substantially as shown in Table 1 or Table 2 (provided in Example 3). In a further embodiment, the crystalline form is characterised by an XRPD pattern substantially as shown in FIG. 1B.

In another embodiment, the crystalline form is characterised by a differential scanning calorimetry (DSC) curve comprising an endotherm at about (e.g. ±5, ±3, ±2 or ±1) 92° C. when measured at a heating speed of 10° C./min. Preferably, the crystalline form is characterised by a DSC curve substantially as shown in FIG. 2.

A further embodiment of the invention provides a pharmaceutical composition comprising a crystalline form of the compound of formula (Ia) and a pharmaceutically acceptable excipient.

In a further embodiment, the invention provides a method for the preparation of a pharmaceutical composition. The method comprises combining a crystalline form of the compound of formula (Ia) and a pharmaceutically acceptable excipient.

In another embodiment, the invention provides a method for the preparation of a crystalline form of the compound of formula (Ia). In a first embodiment, the method comprises:

(a) mixing (i) a composition comprising an amorphous form of a compound of formula (Ia) which is not adsorbed on one or more carrier particles and (ii) a composition comprising a compound of formula (Ia) which is adsorbed on one or more carrier particles, with a suitable solvent;

(b) maintaining the resulting mixture under conditions suitable to provide the crystalline form of the compound of formula (Ia) of the invention; and optionally

(c) removing the solvent.

In a second embodiment, the method comprises:

(a) mixing (i) a composition comprising an amorphous form of a compound of formula (Ia) which is not adsorbed on one or more carrier particles and (ii) a seed of the crystalline form as described herein, with a suitable solvent;

(b) maintaining the resulting mixture under conditions suitable to provide crystalline form of the invention; and optionally

(c) removing the solvent.

In particular embodiments of the second method, in step (a) the amount of seed is about 0.5 to about 10% by weight of the amount of the amorphous compound of formula (Ia) which is not adsorbed on one or more carrier particles.

The one or more carrier particles may be selected from the group consisting of kaolin, bentonite, hectorite, colloidal magnesium-aluminum silicate, silicon dioxide, magnesium trisilicate, aluminum hydroxide, magnesium hydroxide, magnesium oxide or talc. Typically, the one or more carrier particles is silicon dioxide, preferably fumed silicon dioxide.

In particular embodiments of the above methods, the suitable solvent comprises one or more of methyl-tert-butyl ether, toluene, isopropyl alcohol, ethyl alcohol, 2-methyltetrahydrofuran, acetonitrile, dimethylsulfoxide, n-butanol, ethyl acetate, isopropyl acetate, N,N-dimethylformamide, acetone, n-heptane, heptanes, N-methyl-2-pyrrolidinone and water. Typically, the suitable solvent comprises methyl-tent-butyl ether.

In particular embodiments of the above methods, step (b) is carried out at a temperature in the range of from about 5° C. to about 50° C., preferably from about 15° C. to about 25° C.

In particular embodiments of the above methods, step (b) comprises agitation for at least about 12 hours, preferably for at least about 12 hours to about 36 hours.

Also provided is a crystalline form obtained by the methods described herein.

In another embodiment, the invention provides a method comprising administration of a crystalline form of the compound of formula (Ia) to a subject. A particular embodiment provides a method for inhibiting the activity of cytochrome P-450 monooxygenase in a subject comprising administering an effective amount of a crystalline form or a pharmaceutical composition described herein to the subject.

Another embodiment provides a method for the prophylactic or therapeutic treatment of an HIV infection in a subject comprising administering a pharmaceutical composition comprising an effective amount of the crystalline form described herein, or a pharmaceutical composition described herein, to the subject.

Also provided is a crystalline form or a pharmaceutical composition described herein for use in therapy. Also provided is a crystalline form or a pharmaceutical composition described herein for use in inhibiting the activity of cytochrome P-450 monooxygenase in a subject. Also provided is a crystalline form or a pharmaceutical composition described herein for use in a method for the prophylactic or therapeutic treatment of an HIV infection.

A further embodiment provides the use of a crystalline form described herein in the manufacture of a medicament for the prophylactic or therapeutic treatment of an HIV infection.

### BRIEF DESCRIPTION OF DRAWINGS

FIG. 1 is an XRPD pattern of the crystalline form of the compound of formula (Ia) when wet (FIG. 1A) and when dry (FIG. 1B).

FIG. 2 is a DSC curve of a crystalline form of the compound of formula (Ia).

FIG. 3 is a TGA profile of a crystalline form of the compound of formula (Ia).

FIG. 4 is a ¹H NMR spectrum for a crystalline form of the compound of formula (Ia).

### DETAILED DESCRIPTION

Compound of Formula (Ia)

The compound of formula (Ia) (cobicistat, COBI, C, GS-9350) is an inhibitor of cytochrome P-450 3A enzymes. It has the following formula:

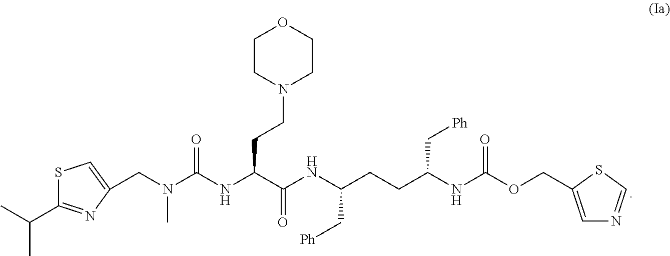 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

Its chemical name is 1,3-thiazol-5-ylmethyl [(2R,5R)-5-{[(2S)-2-[(methyl{[2-(propan-2-yl)-1,3-thiazol-4-yl]methyl}carbamoyl)amino]-4-(morpholin-4-yl)butanoyl]amino}-1,6-diphenylhexan-2-yl]carbamate. It has been authorised as part of STRIBILD® (elvitegravir 150 mg, cobicistat 150 mg, emtricitabine 200 mg, tenofovir disoproxil fumarate 300 mg equivalent to 245 mg tenofovir disoproxil), TYBOST® (cobicistat 150 mg), REZOLSTA® (darunavir (as ethanolate) 800 mg, cobicistat 150 mg), EVOTAZ® (atazanavir 300 mg, cobicistat 150 mg) and GENVOYA® (elvitegravir 150 mg, cobicistat 150 mg, emtricitabine 200 mg, tenofovir alafenamide fumarate (in the hemifumarate form) equivalent to 10 mg tenofovir alafenamide).

In the above existing products, cobicistat is an amorphous solid which is adsorbed on silicon dioxide. Compositions in which cobicistat is adsorbed on silicon dioxide are described in WO 2009/135179.

Manufacturing Methods

In some embodiments, the crystalline form of the invention may be prepared by the following method:

(a) mixing (i) a composition comprising an amorphous form of a compound of formula (Ia) which is not adsorbed on one or more carrier particles and (ii) a seed of the crystalline form of the invention, with a suitable solvent;

(b) maintaining the resulting mixture under conditions suitable to provide crystalline form of the invention; and optionally

(c) removing the solvent.

The suitable solvent is any solvent that yields the crystalline form of the invention when used in the above method. Preferably, the solvent comprises one or more of methyl-tert-butyl ether, toluene, isopropyl alcohol, ethyl alcohol, 2-methyltetrahydrofuran, acetonitrile, dimethylsulfoxide, n-butanol, ethyl acetate, isopropyl acetate, N,N-dimethylformamide, acetone, n-heptane, heptanes, N-methyl-2-pyrrolidinone and water. Typically, the suitable solvent comprises methyl-tert-butyl ether.

The concentration of the amorphous form of the compound of formula (Ia) in the suitable solvent may be in the range from 50-500 mg/mL, preferably, 50-200 mg/mL, most preferably 80-150 mg/mL.

In step (a) of the above method the amount of seed may be from about 0.01 to about 10% by weight of the amount of the amorphous compound of formula (Ia) which is not adsorbed on one or more carrier particles, such as from about 0.1 to about 5% by weight.

In step (a) of the above method, (i) a composition comprising an amorphous form of a compound of formula (Ia) which is not adsorbed on one or more carrier particles and (ii) a seed of the crystalline form of the invention may be present in combination prior to addition to the suitable solvent. Alternatively, the (i) a composition comprising an amorphous form of a compound of formula (Ia) which is not adsorbed on one or more carrier particles and (ii) a seed of the crystalline form of the invention may be added separately to the suitable solvent and then mixed.

Step (b) of the above method may be carried out at a temperature in the range of from about 5° C. to about 50° C., preferably from about 15° C. to about 25° C., e.g. about 20° C. In particular embodiments of the above methods, step (b) comprises agitation. Agitation may be performed for at least about 2 hours, preferably for at least about 12 hours, such as for at least about 12 hours to about 36 hours.

In step (c), removal of the solvent may be by any suitable method known in the art, for example by filtration, by heating, and/or by vacuum drying etc.

Alternative Method

In other embodiments, the crystalline form of the invention may be prepared by the following method:

(a) mixing (i) a composition comprising an amorphous form of a compound of formula (Ia) which is not adsorbed on one or more carrier particles and (ii) a composition comprising a compound of formula (Ia) which is adsorbed on one or more carrier particles, with a suitable solvent;

(b) maintaining the resulting mixture under conditions suitable to provide the crystalline form of the compound of formula (Ia) of the invention; and optionally

(c) removing the solvent.

As for the previous methods, the suitable solvent is any solvent that yields the crystalline form of the invention when used in the above method. Preferably, the solvent comprises one or more of methyl-tent-butyl ether, toluene, isopropyl alcohol, ethyl alcohol, 2-methyltetrahydrofuran, acetonitrile, dimethylsulfoxide, n-butanol, ethyl acetate, isopropyl acetate, N,N-dimethylformamide, acetone, n-heptane, heptanes, N-methyl-2-pyrrolidinone and water. Typically, the suitable solvent comprises methyl-tent-butyl ether.

The concentration of the amorphous form of the compound of formula (Ia) in the suitable solvent may be in the range from 50-500 mg/mL, preferably, 50-200 mg/mL, most preferably 80-150 mg/mL

Step (b) of the above method may be carried out at a temperature in the range of from about 5° C. to about 50° C., preferably from about 15° C. to about 25° C., e.g. about 20° C. In particular embodiments of the above methods, step (b) comprises agitation. Agitation may be performed for at least about 12 hours, preferably for at least about 12 hours to about 36 hours.

In step (c), removal of the solvent may be by any suitable method known in the art, for example by filtration, by heating, and/or by vacuum drying etc.

### Specific Embodiments of the Invention

Specific embodiments identified herein are for illustration; they do not in any way exclude other embodiments of the invention.

The invention also provides a composition comprising the compound of formula (Ia), wherein at least about 0.1% of the compound of formula (Ia) in the composition is present in the crystalline form of the invention. Typically, at least about (a) 5%, (b) 10%, (c) 20%, (d) 30%, (e) 40%, (f) 50%, (g) 60%, (h) 70%, (i) 80%, (j) 85%, (k) 90%, (l) 95%, (m) 99%, (n) 99.5% or (o) 99.9% of the compound of formula (Ia) in the composition is present in the crystalline form of the invention. In some embodiments, at least 95% of the compound of formula (Ia) in the composition is present in the crystalline form of the invention. Where another form of the compound of formula (Ia) is present in the composition, this other form will typically be the amorphous form.

The composition may further comprise one or more carrier particles. In particular, at least about (a) 5%, (b) 10%, (c) 20%, (d) 30%, (e) 40%, (f) 50%, (g) 60%, (h) 70%, (i) 80%, (j) 85%, (k) 90%, (l) 95%, (m) 99%, (n) 99.5% or (o) 99.9% of the compound of formula (Ia) in the composition may be adsorbed on the one or more carrier particles.

The one or more carrier particles may be selected from the group consisting of kaolin, bentonite, hectorite, colloidal magnesium-aluminum silicate, silicon dioxide, magnesium trisilicate, aluminum hydroxide, magnesium hydroxide, magnesium oxide and talc. Typically, the one or more carrier particles is silicon dioxide. Where compositions contain one or more carrier particles, the weight ratio of the compound of formula (Ia) to the one or more carrier particles may be about 1:1.

The composition may be produced by heating in a suitable solvent a compound of formula (Ia) which is adsorbed on one or more carrier particles as described herein. Typically, the suitable solvent is heptane or methyl-tert-butyl ether and the one or more carrier particles is silicon dioxide.

The pharmaceutical compositions of the invention comprise the crystalline form or compositions described herein, in addition to a pharmaceutically acceptable excipient.

The pharmaceutical compositions of the invention may contain about 5 to 500 mg, about 50 to 250 mg, or about 100 to 200 mg of the compound of formula (Ia). A preferred amount for the compound of formula (Ia) in a pharmaceutical composition is 150 mg.

Pharmaceutical Formulations

For pharmaceutical use, the compounds of the invention may be administered as a medicament by enteral or parenteral routes, including intravenous, intramuscular, subcutaneous, transdermal, airway (aerosol), oral, intranasal, rectal, vaginal and topical (including buccal and sublingual) administration. Oral administration is most typical.

Generally, the crystalline form of the invention will be administered as a pharmaceutical composition that comprises one or more pharmaceutically acceptable excipients. Excipients should be compatible with the other ingredients of the formulation and physiologically innocuous to the recipient thereof. Examples of suitable excipients are well known to the person skilled in the art of tablet formulation and may be found e.g. in Handbook of Pharmaceutical Excipients (eds. Rowe, Sheskey & Quinn), 6th edition 2009. As used herein the term “excipient” is intended to refer to inter alia basifying agents, solubilisers, glidants, fillers, binders, lubricant, diluents, preservatives, surface active agents, dispersing agents and the like. The term also includes agents such as sweetening agents, flavouring agents, colouring agents and preserving agents. The choice of excipient will to a large extent depend on factors such as the particular mode of administration, the effect of the excipient on solubility and stability, and the nature of the dosage form.

Typical pharmaceutically acceptable excipients include:

diluents, e.g. lactose, dextrose, sucrose, mannitol, sorbitol, cellulose and/or glycine;

lubricants, e.g. silica, talcum, stearic acid, its magnesium or calcium salt and/or polyethyleneglycol;

binders, e.g. magnesium aluminum silicate, starch paste, gelatin, tragacanth, methylcellulose, sodium carboxymethylcellulose and/or polyvinylpyrrolidone;

disintegrants, e.g. starches, agar, alginic acid or its sodium salt, or effervescent mixtures; and/or

absorbants, colorants, flavors and/or sweeteners.

A thorough discussion of pharmaceutically acceptable excipients is available in Gennaro, Remington: The Science and Practice of Pharmacy 2000, 20th edition (ISBN: 0683306472).

Preferably, the pharmaceutical composition is a solid dosage form suitable for oral administration, such as a tablet or capsule. Tablets are particularly preferred.

Formulations suitable for oral administration may be designed to deliver the crystalline form of the invention in an immediate release manner or in a rate-sustaining manner, wherein the release profile can be delayed, pulsed, controlled, sustained, or delayed and sustained or modified in such a manner which optimises the therapeutic efficacy. Means to deliver compounds in a rate-sustaining manner are known in the art and include slow release polymers that can be formulated with the said compounds to control their release.

The formulation of tablets is discussed in H. Lieberman and L. Lachman, Pharmaceutical Dosage Forms: Tablets 1980, vol. 1 (Marcel Dekker, New York).

Therapeutic Methods

The invention provides a method for the prophylactic or therapeutic treatment of an HIV infection in a subject, comprising administering an effective amount of the crystalline form of the invention to the subject along with another agent.

The invention also provides a method for improving the pharmacokinetics of a drug which is metabolized by cytochrome P450 monooxygenase (e.g. cytochrome P450 monooxygenase 3A), comprising administering to a subject undergoing treatment with said drug, an effective amount of the crystalline form of the invention.

In another embodiment, the present invention provides a method for increasing blood plasma levels of a drug which is metabolized by cytochrome P450 monooxygenase (e.g. cytochrome P450 monooxygenase 3A), comprising administering to a subject undergoing treatment with said drug, an effective amount of the crystalline form of the invention.

In yet another embodiment, the present application provides a method for inhibiting cytochrome P450 monooxygenase (e.g. cytochrome P450 monooxygenase 3A) in a subject comprising administering to a subject an effective amount of the crystalline form of the invention.

The invention provides a crystalline form of the invention for use in any of the above therapeutic methods. Also provided is the use of a crystalline form of the invention for the manufacture of a medicament for use in the above therapeutic methods. Also provided is a crystalline form of the invention for use in therapy.

Compositions of the invention are preferably suitable to be administered once daily, but may be suitable for administration at other dosing frequencies depending on the disease state, patient etc. For example, compositions of the invention may be administered one, two, three or four times per day, or less frequently than once per day.

General

References to the “crystalline form of the invention” mean a crystalline form of the compound of formula (Ia). While crystalline forms are non-amorphous, they may be in a composition comprising amorphous material.

The term “comprise” and variations thereof, such as “comprises” and “comprising”, are to be construed in an open, inclusive sense, i.e. as “including, but not limited to”.

The term “between” with reference to two values includes those two values e.g. the range “between” 10 mg and 20 mg encompasses e.g. 10, 11, 12, 13, 14, 15, 16, 17, 18, 19 and 20 mg.

The term “about” in relation to a numerical value x is optional and, unless otherwise specified, means, for example, x±10%, x±5%, or x±1%.

The term “about” in relation to the position p of a peak (degrees 2θ) in a XRPD spectrum is optional and, unless otherwise specified, means p±0.5, p±0.3, p±0.2, p±0.1, or p±0.05. In particular embodiments, the term about means p±0.1.

Reference throughout this specification to “one embodiment” or “an embodiment” means that a particular feature, structure or characteristic described in connection with the embodiment is included in at least one embodiment provided herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “pharmaceutically acceptable” with respect to a substance refers to that substance which is generally regarded as safe and suitable for use without undue toxicity, irritation, allergic response, and the like, commensurate with a reasonable benefit/risk ratio.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g. an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

As used herein, the term “salts” includes co-crystals. The term “co-crystal” refers to a crystalline compound comprising two or more molecular components, e.g. wherein proton transfer between the molecular components is partial or incomplete.

The term “amorphous” or “amorphous form” refers to a non-crystalline solid form. While amorphous forms are non-crystalline, they may be in a composition comprising crystalline material.

The term “solvate” means a molecular complex comprising a compound and one or more pharmaceutically acceptable solvent molecules. Examples of solvent molecules include water and C₁₋₆ alcohols, e.g. ethanol. When the solvate is water, the term “hydrate” may be used.

“Treating” and “treatment” of a disease include the following:

(1) preventing or reducing the risk of developing the disease, i.e. causing the clinical symptoms of the disease not to develop in a subject that may be exposed to or predisposed to the disease but does not yet experience or display symptoms of the disease,

(2) inhibiting the disease, i.e. arresting or reducing the development of the disease or its clinical symptoms, and

(3) relieving the disease, i.e. causing regression of the disease or its clinical symptoms.

The term “effective amount” refers to an amount that may be effective to elicit the desired biological or medical response, including the amount of a compound that, when administered to a subject for treating a disease, is sufficient to effect such treatment for the disease. The effective amount may vary depending on the compound, the disease and its severity and the age, weight, etc. of the subject to be treated. Useful dosages of can be determined by comparing their in vitro activity, and in vivo activity in animal models. Methods for the extrapolation of effective dosages in mice, and other animals, to humans are known to the art.

### EXAMPLES

The invention will now be illustrated by the following non-limiting examples.

General Experimental Details

XRPD (X-ray Powder Diffraction) analysis was conducted on PANanalytical X'PERT-PRO (PANalytical B. V., Almelo, Netherlands) using copper radiation (Cu Kα, λ=1.5418 Å). Samples were prepared for analysis by depositing the wet cake or powder sample in the center of an aluminum holder equipped with a zero background plate (25 mm diameter). The X-ray generator was operated at a voltage of 45 kV and amperage of 40 mA. The sample rotation speed during measurement was 2 seconds/revolution. Scans were performed from 2 to 40° 2-theta range. The step size was 0.008° and total scan time was 1 hour. Diffraction data was analyzed by X'Pert Highscore version 2.2c (PANalytical B. V., Almelo, Netherlands) and X'Pert data viewer version 1.2d (PANalytical B. V., Almelo, Netherlands)

DSC (Differential Scanning Calorimetry) data were collected on a TA Instruments Q2000 system equipped with a 50 position auto-sampler. The calibration for energy and temperature was carried out using certified indium. The sample was placed into an aluminum DSC pan, and the weight accurately recorded. Typically 2-10 mg of each sample was placed into an aluminium pan. The pan was covered with a lid, then crimped or hermetically sealed or left unsealed. The sample pan was then heated in the DSC cell at rate of 10° C./min up to a final temperature of 300° C. with a dry nitrogen purge rate of 50 mL/min maintained over the sample throughout the measurement.

TGA (Thermogravimetric Analysis) data were collected using a TA Instruments Q5000 TGA instrument equipped with a 25 position auto-sampler. The TGA furnace was calibrated using the magnetic Curie point method. Typically 5-20 mg of sample was loaded onto a pre-tared aluminium pan and heated at 10° C./min to a final temperature of 300° C. with a dry nitrogen purge rate of 25 mL/min maintained over the sample throughout the measurement.

¹H NMR (Proton Nuclear Magnetic Resonance): ¹H NMR spectra were recorded on a Varian 400-MR 400 MHz instrument with 7620AS sample changer. The default proton parameters are as follows: spectral width: 14 to −2 ppm (6397.4 Hz); relaxation delay: 1 sec; pulse: 45 degrees; acquisition time: 2.049 sec; number of scans or repetitions: 8; temperature: 25° C. Samples were prepared in Methanol-d4. Off-line analysis was carried out using MNova software.

### Example 1

MTBE (1 mL) was added to cobicistat (1.0 g) as an amorphous solid in a vial and stirred to mix. To the resulting mixture, about 10 mg cobicistat on silicon dioxide was added. The mixture was mixed in a shaker at room temperature. The mixture was sonicated for multiple cycles to facilitate dissolution of cobicistat and nucleation of cobicistat crystals. The mixture was mixed in shaker for ˜2 weeks to afford a thick paste comprising the crystalline form of the invention.

### Example 2

60 mL MTBE was added to amorphous cobicistat (5 g) in a reaction vessel and stirred. To this mixture was added about 10 mg solid seeds (neat, crystalline cobicistat obtained by the method of Example 1) and agitation was continued overnight. The resulting thick slurry was filtered and the wet cake washed twice with 20 mL MTBE. The wet cake was dried in a desiccator at room temperature under mild vacuum to afford the crystalline form of the invention.

### Example 3

XRPD

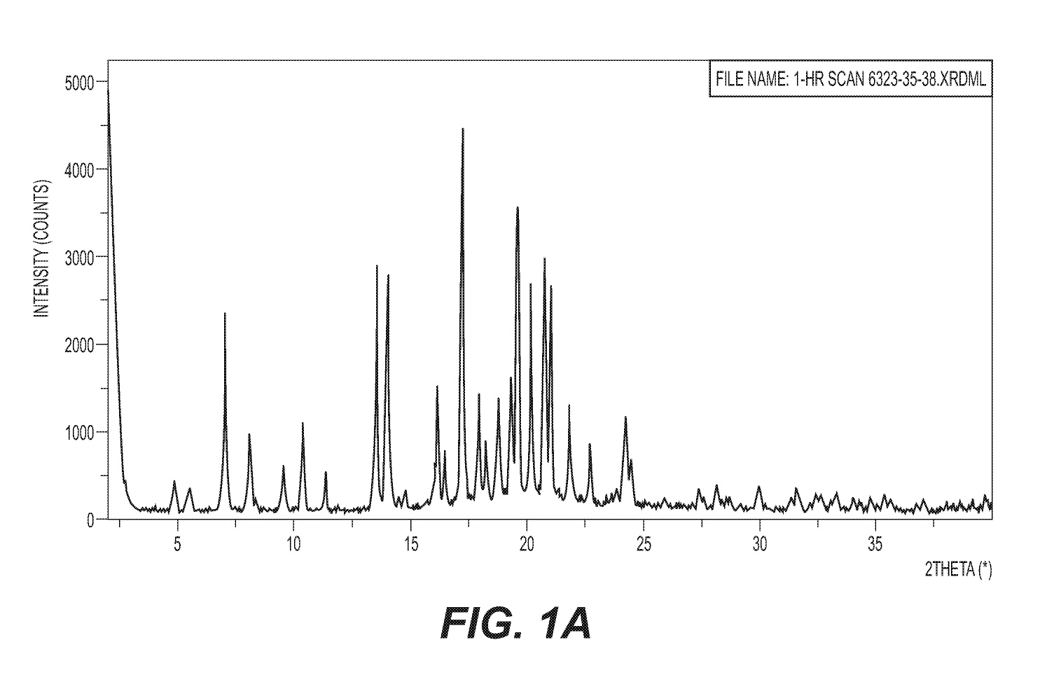

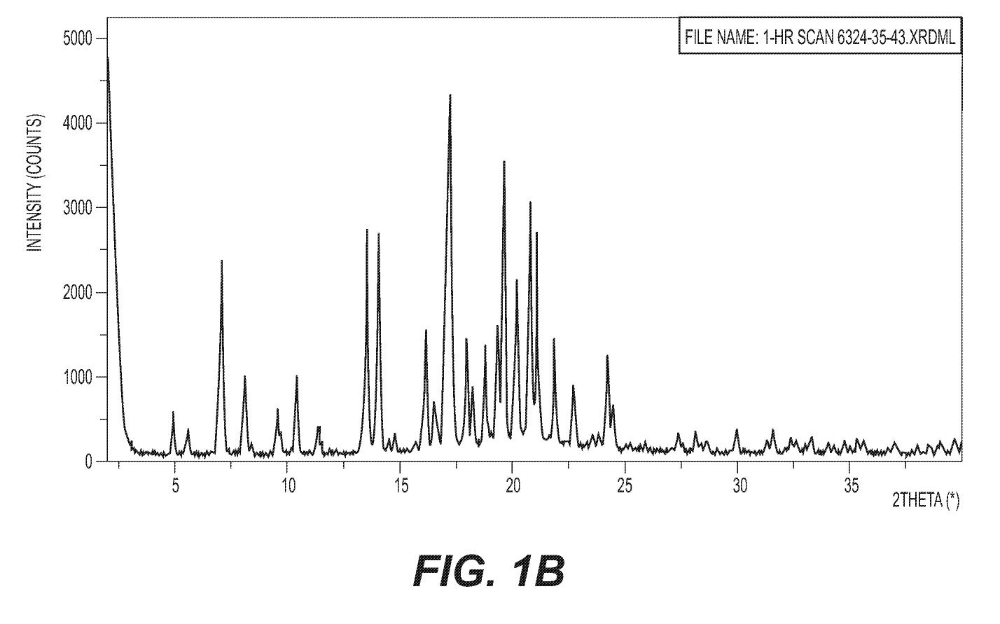

The XRPD pattern of the crystalline form of the invention when wet is shown in FIG. 1A. The XRPD pattern of the crystalline form of the invention when dry is shown in FIG. 1B. The sharp, well-resolved peaks in the XRPD data suggest the material is crystalline.

The positions and intensities of the characteristic peaks observed in the XRPD spectrum are provided in Table 1.

TABLE 1

| | Pos. | Rel. Int. |
|---|---|---|
| No. | [°2Th.] | [%] |
| 1 | 7.028 | 57.18 |
| 2 | 13.5349 | 65.61 |
| 3 | 14.0371 | 62.52 |
| 4 | 17.2211 | 100 |
| 5 | 19.6102 | 79.42 |
| 6 | 20.1581 | 43.75 |
| 7 | 20.7805 | 64.34 |
| 8 | 21.0486 | 60.82 |

The positions of all peaks observed in the XRPD spectrum are provided in Table 2.

TABLE 2

| | Pos. | Rel. Int. |
|---|---|---|
| No. | [°2Th.] | [%] |
| 1 | 4.8753 | 8.35 |
| 2 | 5.5339 | 6.12 |
| 3 | 7.028 | 57.18 |
| 4 | 8.108 | 21.42 |
| 5 | 9.5303 | 12.05 |
| 6 | 10.3824 | 22.33 |
| 7 | 11.353 | 6.92 |
| 8 | 13.5349 | 65.61 |
| 9 | 14.0371 | 62.52 |
| 10 | 14.4802 | 2.76 |
| 11 | 14.7867 | 4.71 |
| 12 | 16.1619 | 31.42 |
| 13 | 16.484 | 12.89 |
| 14 | 17.2211 | 100 |
| 15 | 17.9492 | 28.93 |
| 16 | 18.2369 | 15.07 |
| 17 | 18.7727 | 28 |
| 18 | 19.3527 | 31.95 |
| 19 | 19.6102 | 79.42 |
| 20 | 20.1581 | 43.75 |
| 21 | 20.7805 | 64.34 |
| 22 | 21.0486 | 60.82 |
| 23 | 21.8417 | 23.78 |
| 24 | 22.7305 | 16.82 |
| 25 | 23.8532 | 2.9 |
| 26 | 24.2294 | 24.09 |
| 27 | 24.4925 | 11.32 |
| 28 | 25.9379 | 1.65 |
| 29 | 27.3926 | 4.94 |
| 30 | 28.1603 | 5.66 |
| 31 | 28.7093 | 2.34 |
| 32 | 29.9613 | 5.93 |
| 33 | 31.3324 | 2.74 |
| 34 | 31.6299 | 5.15 |
| 35 | 32.4082 | 3.24 |
| 36 | 33.3429 | 3.64 |
| 37 | 34.0596 | 2.36 |
| 38 | 34.7844 | 3.02 |
| 39 | 35.3733 | 3.39 |
| 40 | 35.6539 | 2.53 |
| 41 | 36.9824 | 1.34 |
| 42 | 38.1013 | 0.86 |
| 43 | 38.6514 | 1.06 |
| 44 | 39.1559 | 1.1 |
| 45 | 39.6795 | 1.59 |

DSC

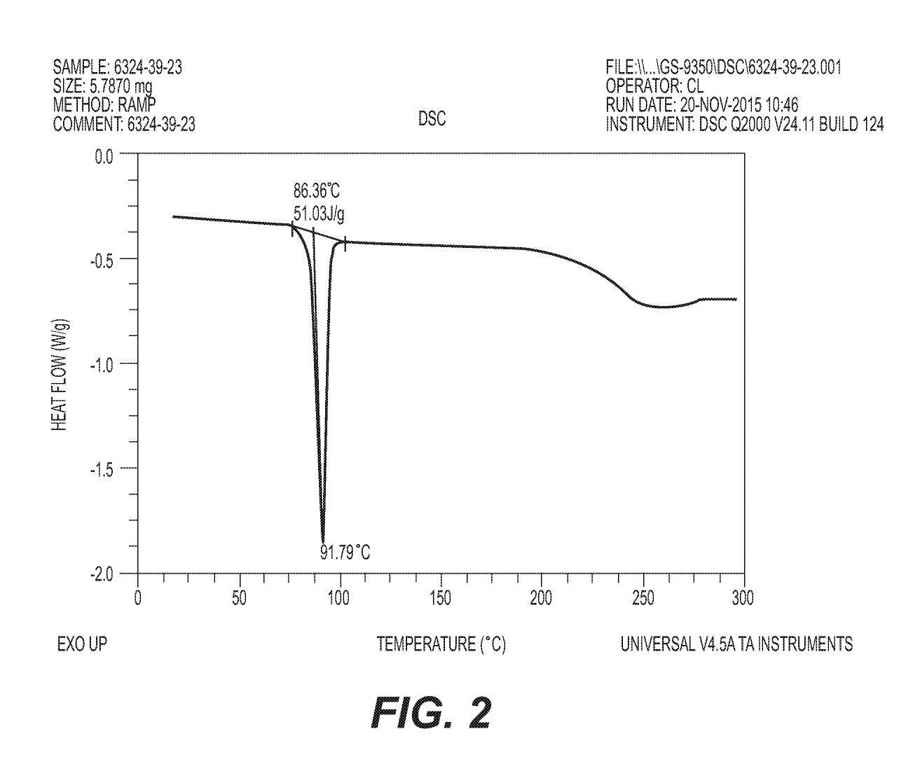

The DSC curve is shown in FIG. 2 and comprises a single endotherm with melting point ca. 90° C.

TGA

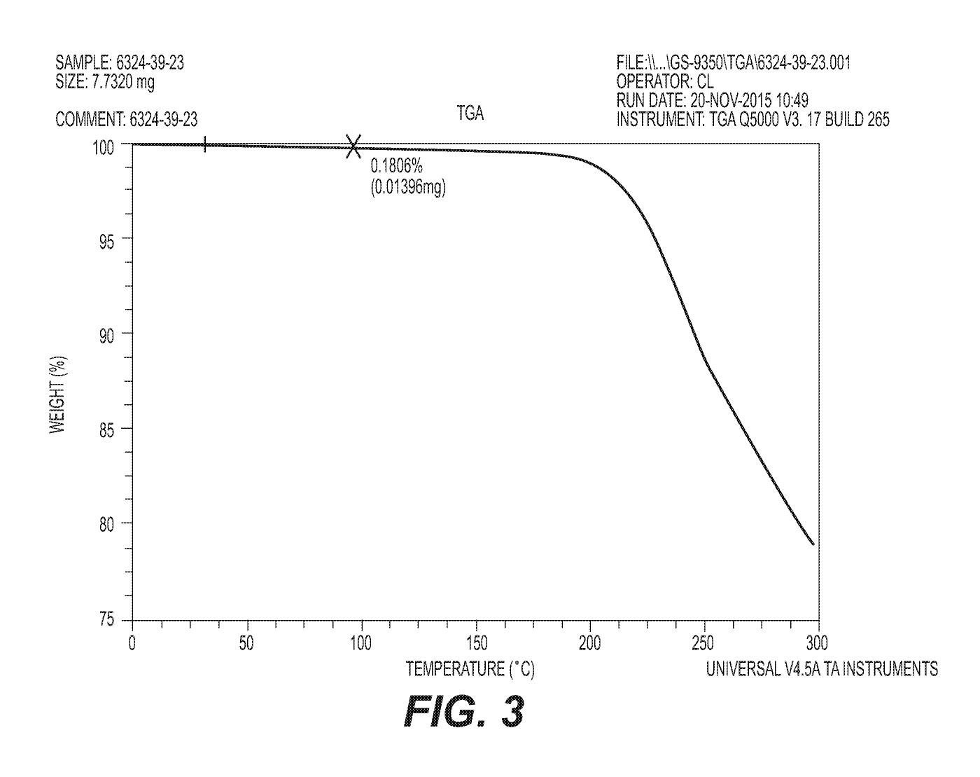

The TGA profile is shown in FIG. 3. The TGA profile shows no solvent loss up to 150° C., indicating that the crystalline form of the invention is an anhydrous, non-solvated form.

NMR

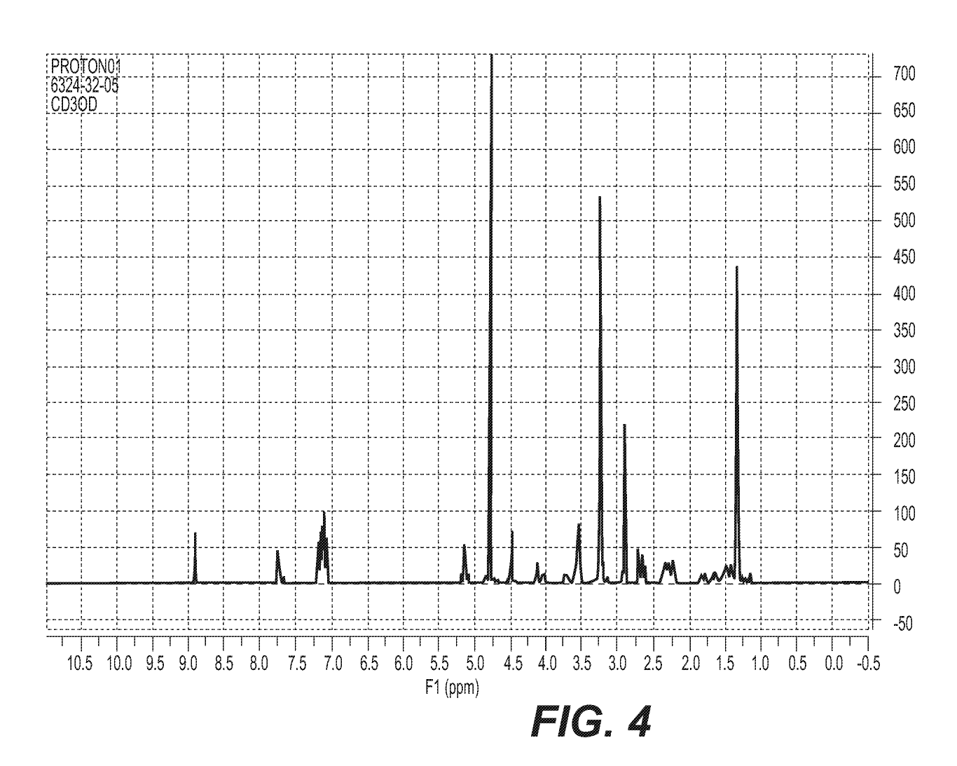

The ¹H NMR spectrum is shown in FIG. 4. The ¹H NMR spectrum is consistent with that of the cobicistat API (amorphous form).

The invention has been described with reference to various specific and preferred embodiments and techniques. However, it should be understood that many variations and modifications may be made while remaining within the spirit and scope of the invention.

1. A crystalline form of a compound of formula (Ia)

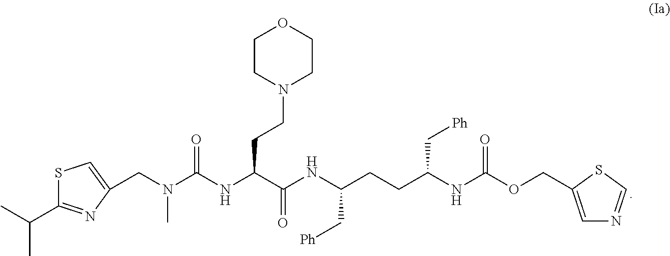 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

2. The crystalline form of claim 1, characterised by an X-ray powder diffraction (XRPD) pattern comprising peaks at 17.2±0.2 and 19.6±0.2 (Cu Kec radiation, expressed in degrees 2θ).

3. The crystalline form of claim 1, characterised by an X-ray powder diffraction (XRPD) pattern comprising peaks at 13.5±0.2, 17.2±0.2, 19.6±0.2 and 20.8±0.2 (Cu Kα radiation, expressed in degrees 2θ).

4. The crystalline form of claim 1, characterised by an X-ray powder diffraction (XRPD) pattern comprising peaks at 7.0±0.2, 13.5±0.2, 14.0±0.2, 17.2±0.2, 19.6±0.2, 20.2±0.2, 20.8±0.2 and 21.0±0.2 (Cu Kα radiation, expressed in degrees 2θ).

5. The crystalline form of claim 1, characterised by a differential scanning calorimetry (DSC) curve comprising an endotherm at 92° C.±3.

6. A composition comprising the compound of formula (Ia), wherein at least about 50% of the compound of formula (Ia) in the composition is present in the crystalline form of claim 1.

7. The composition of claim 6, wherein the composition further comprises one or more carrier particles.

8. The composition of claim 7, wherein at least about 5%, of the compound of formula (Ia) in the composition is adsorbed on the one or more carrier particles.

9. The composition of claim 7, wherein the one or more carrier particles is selected from the group consisting of kaolin, bentonite, hectorite, colloidal magnesium-aluminum silicate, silicon dioxide, magnesium trisilicate, aluminum hydroxide, magnesium hydroxide, magnesium oxide and talc.

10. The composition of claim 9, wherein the one or more carrier particles is silicon dioxide.

11. The composition of claim 7, wherein the weight ratio of the compound of formula (Ia) to the one or more carrier particles is about 1:1.

12. A pharmaceutical composition comprising (i) the crystalline form of claim 1 and a pharmaceutically acceptable excipient.

13. A composition comprising the crystalline form of claim 1, wherein the crystalline form is not adsorbed onto a carrier particle.

14. The composition of claim 13, wherein the carrier particle is silicon dioxide.

15. A method for the preparation of the crystalline form of claim 1 comprising:
(a) mixing (i) a composition comprising an amorphous form of a compound of formula (Ia) which is not adsorbed on one or more carrier particles and (ii) a composition comprising a compound of formula (Ia) which is adsorbed on one or more carrier particles, with a suitable solvent;
(b) maintaining the resulting mixture under conditions suitable to provide the crystalline form of the compound of formula (Ia) of claim 1; and optionally
(c) removing the solvent.

16. A method for the preparation of the crystalline form of claim 1 comprising:
(a) mixing (i) a composition comprising an amorphous form of a compound of formula (Ia) which is not adsorbed on one or more carrier particles and (ii) a seed of the crystalline form of claim 1, with a suitable solvent;
(b) maintaining the resulting mixture under conditions suitable to provide crystalline form of claim 1; and optionally
(c) removing the solvent.

17. The method of 15, wherein the suitable solvent comprises methyl-tent-butyl ether.

18. The method of claim 15, wherein step (b) comprises agitation for at least 12 hours.

19. A method for inhibiting the activity of cytochrome P-450 monooxygenase in a subject comprising administering an effective amount of the crystalline form of claim 1 to the subject.
